(19) United States
(12) Patent Application Publication
(10) Pub. No.: US 2008/0203249 A1
(43) Pub. Date: Aug. 28, 2008

(54) Arm support device

(76) Inventor: David H. Priest, Avon, CT (US)

Correspondence Address: THE LAW OFFICES OF STEVEN MCHUGH, LLC, 46 WASHINGTON STREET, MIDDLETOWN, CT, 06457, US

(21) Appl. No.: 11/710,753

(22) Filed: Feb. 26, 2007

(51) Int. Cl. A61F 5/00

(52) U.S. Cl. 248/118

(57) ABSTRACT

An apparatus for supporting at least one arm of a person performing manual therapy on a patient lying on a table is provided, wherein the apparatus includes a cradle device shaped to supportingly cradle a portion of the arm between a hand portion and an elbow portion of the person. A cradle device support structure is also provided, wherein the cradle device support structure is movably connected to the cradle device such that the cradle device is movable in a plurality of planes relative to at least one of the patient and the table, wherein the cradle device support structure includes a mounting device for connecting the cradle device support structure to a portion of the table, wherein the cradle device support structure is configurable in a plurality of planes relative to the table.

### FIELD OF THE INVENTION

This disclosure relates generally to a device for supporting the arm of a person and more particularly to a device for supporting the arm of a manual therapist performing manual therapy on a patient.

### BACKGROUND OF THE INVENTION

The Craniosacral system is comprised primarily of the Membranes (or Meninges) which surround the major components of the Central Nervous System (hereinafter “CNS”), such as the brain and spinal cord, and is a carefully controlled fluidic environment that has a watertight boundary which is largely provided by the external layer of the meninges, otherwise referred to as the dura mater or dural membrane. The craniosacral system has a controlled fluid inflow that is provided mainly by the choroid plexuses and a controlled fluid outflow that is provided mainly by the arachnoid granulation system. The fluid within the craniosacral system is referred to as cerebrospinal fluid (hereinafter “CSF”), which is extracted from the blood by the choroid plexuses and returned to the blood by the arachnoid granulation system, wherein the extraction and re-absorption of CSF are accomplished largely through osmotic pressures and specialized active transport mechanisms.

As the CSF circulates and fills the spaces between the cells of the brain and the spinal cord, the CSF is allowed to perform several key functions such as, floating the brain to reduce the effects of gravity, serving as a shock absorber to prevent the brain from being traumatized by striking the inner surface of the cranium during sudden movements of the head or body, providing nutrients to the brain and spinal cord, as well as to the pituitary and pineal glands, “washing away” metabolic waste products and toxic substances that have been deposited within and between the cells of the CNS, lubricating between the cells of the CNS to prevent friction or improper contact that could short-circuit electrical nerve impulses or damage cell walls, and maintaining the proper concentration of electrolyte substances (ion) necessary for creating the transmitting nerve impulses to help facilitate brain function. As such, the CSF is a vital and necessary component of the CNS.

As is well known, the dural membrane attaches to the sacrum, the coccyx, the second and third cervical vertebrae, C2 and C3 and all of the bones of the cranium, forming an inner lining (or periosteum) for the cranial bones. As such, nerves and blood vessels to all parts of the body must penetrate the dural membrane as they enter or leave the spinal cord. Thus, any excessive tensions or contractions of the dural membrane can not only constrict the nerves as they enter or leave the membrane throughout its length, but it can compromise the craniosacral system, the CNS and the endocrine, nervous and immune systems. During circulation, the volume of CSF in the craniosacral system constantly rises and falls in a rhythmic fashion at approximately six to twelve cycles per minute. As such, in order to avoid excessive fluid pressures on the delicate structures of the brain and spinal cord, this volumetric change requires that the membrane boundaries continually adapt. In fact, the cranial bones are constantly opening and closing harmoniously with the rise and fall of the CSF. This movement of the cranial bones is essential in properly accommodating the expansion and contraction of the dural membrane as it adjusts to the oscillating volume of CSF. The dural membrane, which is taut and under constant tension, is referred to as a reciprocal tension membrane, indicating that tension on one end of the membrane will be reciprocally transmitted to the other end. So any contraction or excessive tension anywhere in the dural membrane will cause abnormal tensions throughout the entire system. So when there is disease, injury or direct trauma to the head, spine or pelvis, the dural membrane may become twisted and compressed, thus causing ramifications to the CNS, endocrine, nervous and immune systems.

CranioSacral therapy (hereinafter “CST”) is a holistic therapy approach that seeks to aid the circulation of the CSF throughout the CNS and typically involves the manipulation of the membranes that line the nervous system structures, such as the bones of the skull (i.e. the cranium) and the sacrum, to relieve physical pain, restore the natural rhythmic movement found between the bones of the skull and to help treat a variety of other ailments, such as seizures and autism. In light of the above discussion, in order to achieve the most benefit, CST tends to focus on four main targets of wellness. The first target involves impaired mobility of the bones related to the craniosacral system, such as the cranium, the face, the vertebral column and the pelvis. During therapy the therapist attempts to address any factors that contribute to the impaired bone mobility with the goal of restoring as much motion as possible while compromising the craniosacral system function as little as possible. The second target involves the impaired accommodative abilities of the dural membrane system which must be allowed to move freely in the vertebral canal and expand in the cranium. These restrictive factors typically include bone restrictions and abnormal tension patterns within the dural membrane itself which are broadcasted into the cranium from ligamentous and/or muscle attachments that are excessively tight due to adhesions or cohesions formed subsequent to infections, surgical interventions, bleeding and/or toxic depositions.

The third target involves the flow of CSF and blood throughout the craniosacral system. During therapy the goal is to enhance, as much as possible, the flow of CSF and blood. It is imperative that blood flows freely into the choroids plexus and through the arachnoid membrane system in order for the CSF to be formed, reabsorbed and able to perform its function. Thus, because the membranes of the craniosacral system separate blood from CSF many CST techniques focus on CSF circulation, enhancing the blood supply and venous drainage. The fourth target tends to involve the impact of the conscious awareness of the client to the CST in order for the client to “get in touch” with and favorably influence their bodily functions.

During CST the patient is typically disposed in a supine, back down position on a table while the therapist is typically situated near the head and neck region of the patient. Additionally, the therapist may also be situated near the pelvic region as well as other various areas throughout the torso of the patient. Unfortunately, the therapist is frequently disposed such that the hands, wrists and/or fingers of the therapist are positioned in a non-ergonomic position. In fact, the hands, wrists and/or fingers of the therapist are unsupported and frequently bent and contorted and called upon to apply some pressure to the body of the patient for an extended period of time. This is undesirable because this tends to result in injury to the shoulders, wrist, hands and/or forearms of the therapist.

### SUMMARY OF THE INVENTION

An apparatus for supporting at least one arm of a person performing manual therapy on a patient lying on a table is provided, wherein the apparatus includes a cradle device shaped to supportingly cradle a portion of the arm between a hand portion and an elbow portion of the person. A cradle device support structure is also provided, wherein the cradle device support structure is movably connected to the cradle device such that the cradle device is movable in a plurality of planes relative to at least one of the patient and the table, wherein the cradle device support structure includes a mounting device for connecting the cradle device support structure to a portion of the table, wherein the cradle device support structure is configurable in a plurality of planes relative to the table.

An apparatus for supporting at least one arm of a person performing manual therapy, wherein the apparatus includes a patient support table, a cradle device shaped to supportingly cradle a portion of the arm between a hand portion and an elbow portion of the person and a cradle support structure movably connected to the cradle device such that the cradle device is movable in a plurality of planes relative to at least one of the patient and said patient support table, wherein the cradle device support structure includes a mounting device for connecting the cradle device support structure to a portion of the patient support table, wherein the cradle device support structure is configurable in a plurality of planes relative to the patient support table.

### BRIEF DESCRIPTION OF DRAWINGS

The foregoing and other features and advantages of the present invention will be more fully understood from the following detailed description of illustrative embodiments, taken in conjunction with the accompanying drawings in which like elements are numbered alike:

FIG. 1a is a side view of a support apparatus for supporting the arm of a therapist, in accordance with a first embodiment of the present invention;

FIG. 1b is a perspective view showing the relationship between the sagittal plane, the transverse plane and the coronal plane.

FIG. 2a is a side view of the support apparatus of FIG. 1 being connected to a patient support device illustrating a first embodiment of a mounting device, in accordance with the present invention;

FIG. 2b is a side view of the support apparatus of FIG. 1 being connected to a patient support device illustrating a first embodiment of a mounting device, in accordance with the present invention;

FIG. 3a is a side view of the support apparatus of FIG. 1 being connected to a patient support device illustrating a second embodiment of a mounting device, in accordance with the present invention;

FIG. 3b is a side view of the support apparatus of FIG. 1 being connected to a patient support device illustrating a second embodiment of a mounting device, in accordance with the present invention;

FIG. 4 is a side view of the support apparatus of FIG. 1 illustrating one embodiment of the attachment devices, in accordance with the present invention;

FIG. 5 is a side view of the support apparatus of FIG. 1 illustrating one embodiment of the support device portion in an extended configuration, in accordance with the present invention; and

FIG. 6 is a side view of the support apparatus of FIG. 1 illustrating one embodiment of the support device portion in a retracted configuration, in accordance with the present invention.

### DETAILED DESCRIPTION OF THE INVENTION

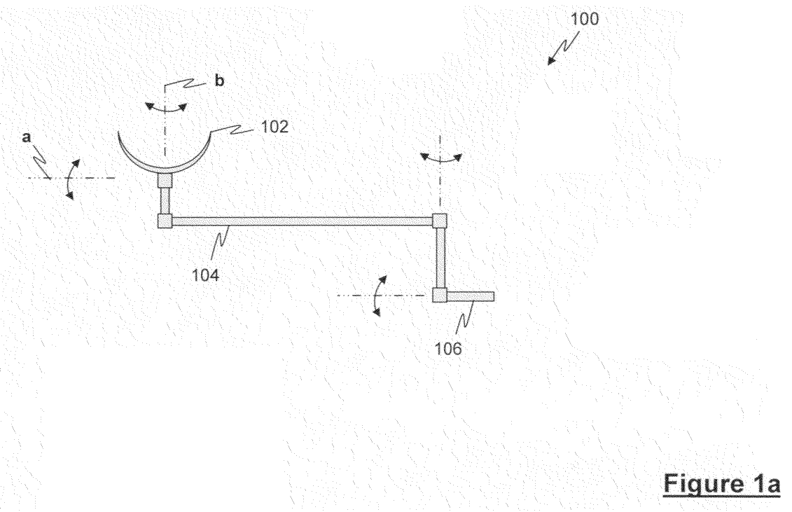

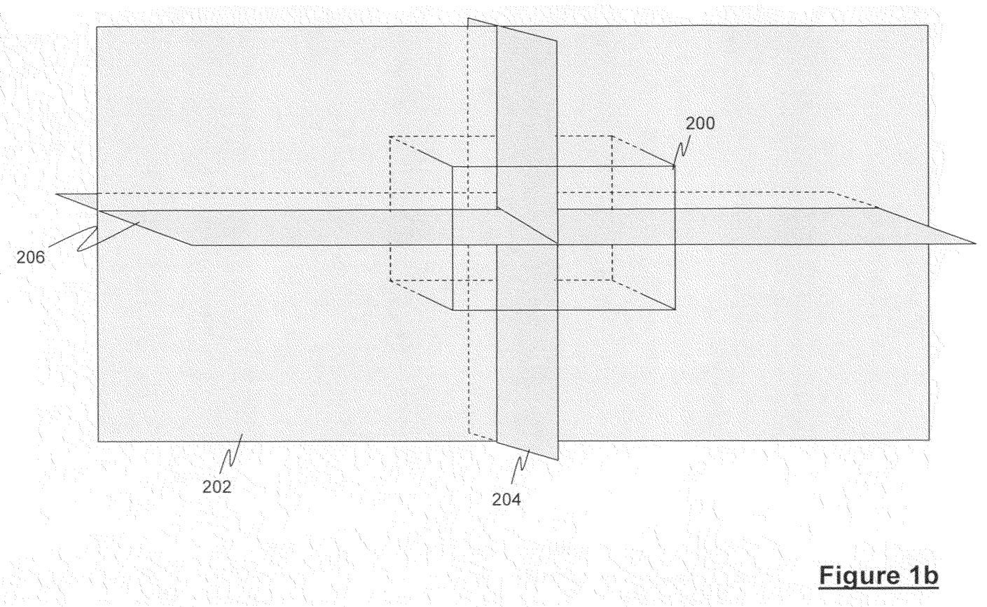

It should be appreciated that although the present invention is described herein as being used applied with craniosacral therapy, the present invention may be applied with any type of manual therapy. Referring to FIG. 1a, a side view of a support apparatus 100 for supporting the arm of a person performing craniosacral therapy on a patient is shown in accordance with a first embodiment. The support apparatus 100 includes a cradle device 102 that is movably and configurably connected to a cradle support device 104 as will be discussed in further detail hereinafter. The cradle support device 104 includes a cradle mounting device 106 for securely connecting the cradle support device 104 to a patient support structure, such as a massage table and a medical examination table. The cradle mounting device 106 is removably associated with the cradle support device 104, as will be discussed in further detail hereinafter, to allow the support apparatus 100 to be configured for a secure connection with a variety of patient support structures. It should be appreciated that the cradle device support 104 is movably connected to the cradle device 102 such that the cradle device 102 is movable in multiple planes, such as two, three, four or more planes.

Thus, referring to FIG. 1b, the support apparatus 104 (represented by box 200) is configurably movable (up to 360°) in at least one of the coronal plane 202, the sagittal plane 204 and the transverse plane 206. Additionally, it is considered within the scope of the invention that the support apparatus 104 may also be movable (up to 360°) about any plane in between the sagittal plane 202, the transverse plane 204 and the coronal plane 206. For example, consider the situation in which the starting position of the arm of the therapist is such that the forearm and hand are parallel to the ground with the palm of the hand facing down. If the first movement takes place on a sagittal or vertical plane, the hand and/or arm (and thus the cradle device 102 and/or cradle support device 104) may move so that the hand and fingers are either tipped down towards the ground, or pointed up towards the sky. If the movement takes place on a coronal plane, the hand and/or arm (and thus the cradle device 102 and/or cradle support device 104) will be able to move (i.e. tilt) side to side where the thumb and radial side of the forearm will either point up towards the sky, or down towards the ground. Lastly, if the movement that takes place on a transverse plane, the arm, hand and/or fingers will move either left or right while they are still parallel to the ground.

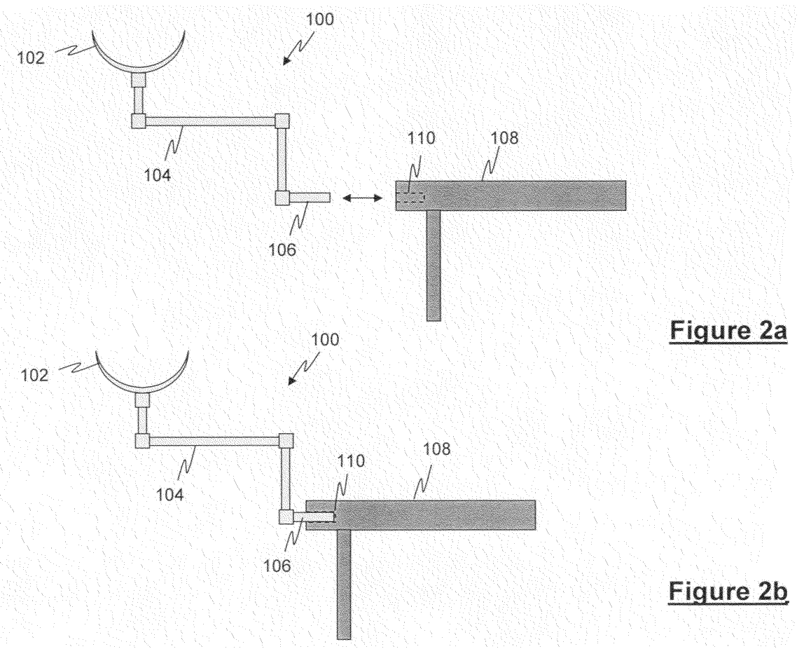

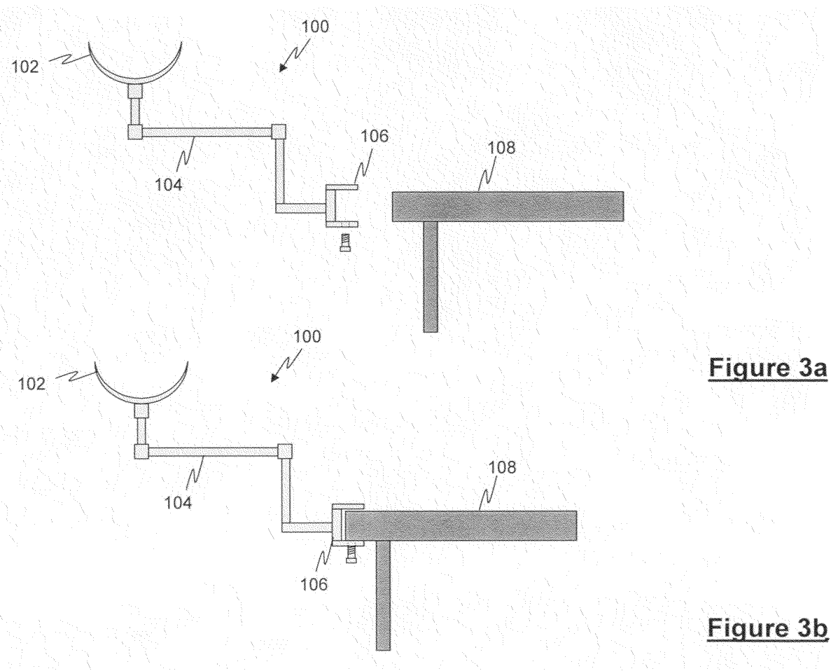

Referring to FIG. 2a and FIG. 2b, if the table being used is a common massage table 108, the cradle mounting device 106 may be a pronged male structure for frictionally interfacing with a female table mounting cavity 110 typically reserved for a head support device. In this situation, the pronged male structure 106 is inserted into and supported by the table mounting cavity 110, wherein when the craniosacral therapy is completed and the support apparatus 100 is no longer required, the pronged male structure 106 may be removed from the table mounting cavity 110 and the support apparatus 100 may be stored for future use. On the other hand, if the table being used does not have the female table mounting cavity typically reserved for a head support device, the cradle mounting device 106 may be a clamping device that frictionally clamps onto a portion of the table, such as the table bed or leg as shown in FIG. 3a and FIG. 3b.

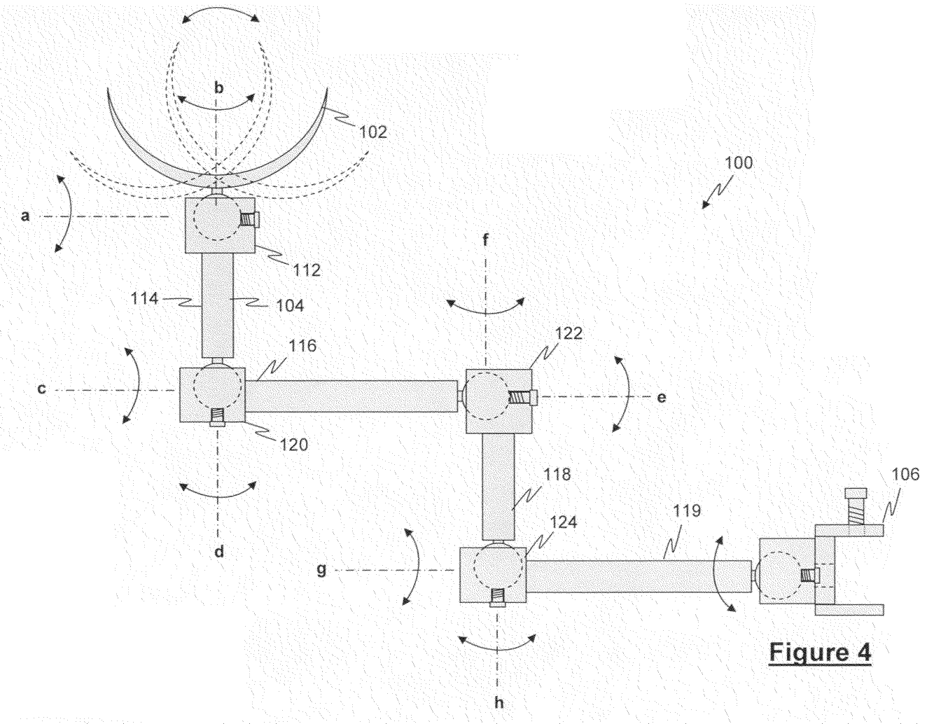

Referring to FIG. 4, a first embodiment of the support apparatus 100 is shown wherein the cradle device 102 is connected to the cradle support device 104 via a cradle attachment device 112 such that the cradle device 102 is movable about a horizontal axis a and a vertical axis b, relative to the cradle attachment device 112. In addition, the cradle support device 104 includes a first support device portion 114, a second support device portion 116, a third support device portion 118 and a fourth support device portion 119, wherein the first support device portion 114 is movably connected to the second support device portion 116 via a first attachment device 120 to allow the first support device portion 114 to be movable about a horizontal axis c and a vertical axis d relative to the first attachment device 120. Additionally, the second support device portion 116 is movably connected to the third support device portion 118 via a second attachment device 122 to allow the second support device portion 116 to be movable about a horizontal axis e and a vertical axis f relative to the second attachment device 122. Furthermore, the third support device portion 118 is movably connected to the fourth support device portion 119 via a third attachment device 124 to allow the third support device portion 118 to be movable about a horizontal axis g and a vertical axis h relative to the third attachment device 124. This allows the support apparatus to be configurable about several different planes simultaneously while providing arm support and more specifically, wrist and/or forearm support, to the therapist.

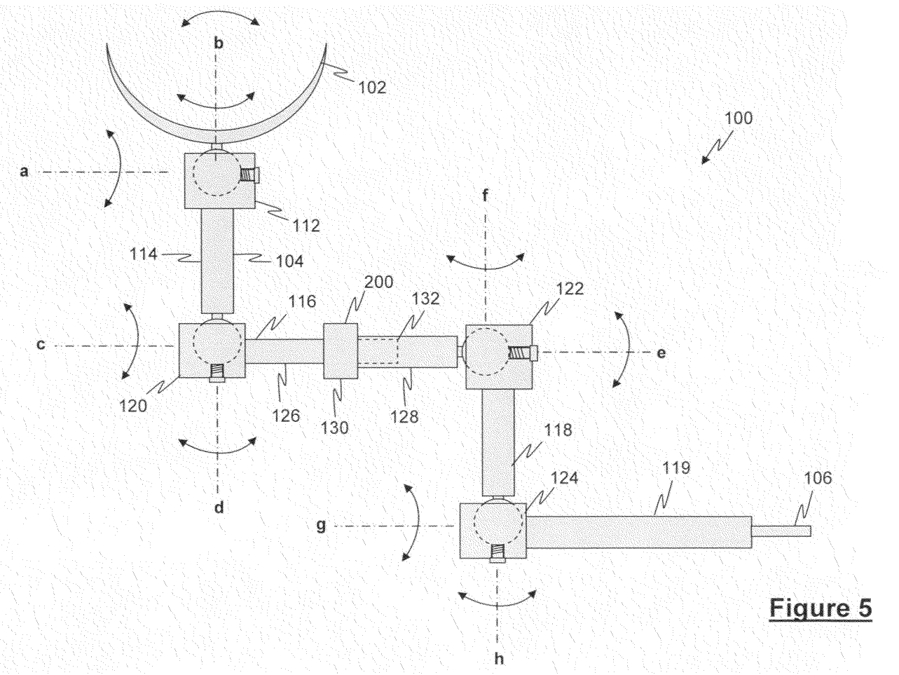

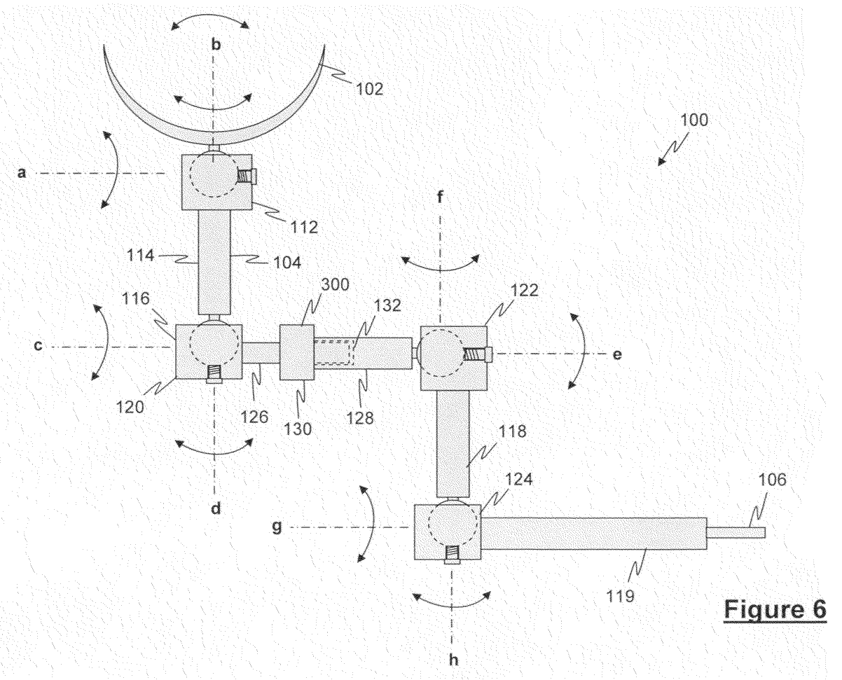

Additionally, it should be appreciated that the support apparatus 100 may include telescoping support device elements 114, 116, 118, 119 as well to allow the therapist to configure the reach of the support apparatus 100. For example, referring to FIG. 5 and FIG. 6, the second support device portion 116 is shown as having a first telescoping portion 126 and a second telescoping portion 128, wherein the second support device portion 116 is configurable between an extended configuration 200 and a retracted configuration 300 via a stabilizing device 130. The stabilizing device 130 may be any type of stabilizing device 130 suitable to the desired end purpose, such as a compression based stabilizing device. In this situation, when the compression based stabilizing device 130 is rotated in one direction, the first telescoping portion 126 is allowed to telescope into and/or out of a containment cavity 132 within the second telescoping portion 128 and when rotated in the opposing direction a portion of the second telescoping portion 128 is directed to compress against the first telescoping portion 126 to prevent the first telescoping portion 126 from moving relative to the second telescoping portion 128. It should also be appreciated that the scope of the present invention contemplates that the first support device portion 114, the third support device portion 118 and/or the cradle mounting device 106 may also have telescoping elements similar to that shown in FIG. 5 and FIG. 6.

It should be appreciated that the support apparatus 100 may be used in situations that require a therapist to maintain his posture in a physically awkward and uncomfortable position for an extended period of time. For example, in accordance with the present invention, one scenario where the support apparatus 100 would be useful may be where a patient receiving craniosacral therapy is lying supine (face up) on the table and the therapist is seated at the side of the patient facing the table. In this situation, the therapist may perform a technique to release any pelvic diaphragm restrictions, where one hand is typically placed under the sacrum and the other hand is typically placed very lightly on top of the pubic bone area. During this process, the therapist is assessing movement and restrictions in this region. While performing this release, the therapist must typically maintain this position for several minutes, and sometimes longer, until a perceived release occurs. If the therapist attempts to hold his arm and hand in this position without any support for an extended period of time, shoulder fatigue will occur. In this situation, the therapist may attach the support apparatus 100 to the side of the table 108 and adjust the apparatus 100 in such a way that his arm and hand is fully supported in an optimum position for performing this technique, thus relieving any wrist, elbow and/or shoulder fatigue that may occur.

Subsequently during a therapy session, the therapist may perform various other techniques to assess and free restrictions of the hard palate, which includes individually addressing the maxilla, vomer, and palatines. This procedure can be very tedious as each bony structure has several movements that must be individually assessed and manipulated. Furthermore, most or all of these techniques require the therapist to be working intra-orally with their hands and fingers. Thus, achieving proper hand and finger placement in these areas are extremely difficult as the wrist usually needs to be awkwardly bent and the shoulder elevated. Fatigue and discomfort of the therapist's wrist and shoulder is very common while performing these techniques. By utilizing the support apparatus 100 to position the therapists arm and wrist in an ergonomically correct way, the therapist can perform the appropriate therapy in a safe, effective, effortless and unobtrusive manner.

While the invention has been described with reference to an exemplary embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

We claim:

1. An apparatus for supporting at least one arm of a person performing manual therapy on a patient lying on a table, the apparatus comprising:
a cradle device shaped to supportingly cradle a portion of the arm between a hand portion and an elbow portion of the person; and
a cradle support structure movably connected to said cradle device such that said cradle device is movable in a plurality of planes relative to at least one of the patient and the table, wherein said cradle device support structure includes a mounting device for connecting said cradle device support structure to a portion of the table, wherein said cradle device support structure is configurable in a plurality of planes relative to the table.

2. The apparatus of claim 1, wherein when portion of the arm between the hand and elbow is disposed on said cradle device, said cradle device is rotatable to approximately 90° from horizontal in a direction toward said hand portion.

3. The apparatus of claim 1, wherein when portion of the arm between the hand and elbow is disposed on said cradle device, said cradle device is rotatable to approximately 90° from horizontal in a direction toward said elbow portion.

4. The apparatus of claim 1, wherein when portion of the arm between the hand and elbow is disposed on said cradle device, said cradle device is rotatable to approximately 180° from horizontal in a side to side direction substantially perpendicular to a line intersecting both of said hand portion and said elbow portion.

5. The apparatus of claim 1, wherein said cradle support device further includes a plurality of support device members.

6. The apparatus of claim 1, wherein said cradle support device further includes a first support device member having a first support device member axis, a second support device member having a second support device member axis, a third support device member having a third support device member axis and a fourth support device member having a fourth support device member axis.

7. The apparatus of claim 6, wherein said first support device member is movably connected to said cradle device and said second support device member, said fourth support device member is movably connected to said mounting device and said third support device member and said second support device member is movably connected to said third support device member.

8. The apparatus of claim 6, wherein said cradle device and said first support device member are movably connected to each other such that said cradle device is movable about said first support device axis.

9. The apparatus of claim 6, wherein said first support device member and said second support device member are movably connected to each other such that said first support device structure is movable about at least one of said first support device axis and said second support device axis.

10. The apparatus of claim 6, wherein said second support device member and said third support device member are movably connected to each other such that said second support device structure is movable about at least one of said second support device axis and said third support device axis.

11. The apparatus of claim 6, wherein said third support device member and said fourth support device member are movably connected to each other such that said third support device structure is movable about at least one of said third support device axis and said fourth support device axis.

12. The apparatus of claim 6, wherein said fourth support device member and said mounting device are movably connected to each other such that said mounting device is movable about said fourth support device axis.

13. The apparatus of claim 1, wherein said mounting device includes a pronged portion for removably interfacing with a common massage table.

14. The apparatus of claim 1, wherein said cradle device is movable in at least one of a sagittal plane, a coronal plane and a transverse plane.

15. The apparatus of claim 14, wherein said cradle device is movable up to approximately 360° in said sagittal plane.

16. The apparatus of claim 14, wherein said cradle device is movable up to approximately 360° in said transverse plane.

17. The apparatus of claim 14, wherein said cradle device is movable up to approximately 360° in said coronal plane.

18. The apparatus of claim 14, wherein at least one of said cradle device and said support device is configurable such that said cradle device is movably positionable a plurality of planes relative to the patient.

19. An apparatus for supporting at least one arm of a person performing manual therapy, the apparatus comprising:
a patient support table;
a cradle device shaped to supportingly cradle a portion of the arm between a hand portion and an elbow portion of the person; and
a cradle support structure movably connected to said cradle device such that said cradle device is movable in a plurality of planes relative to at least one of the patient and said patient support table, wherein said cradle device support structure includes a mounting device for connecting said cradle device support structure to a portion of said patient support table, wherein said cradle device support structure is configurable in a plurality of planes relative to said patient support table.
